(19) United States
(12) Patent Application Publication
(10) Pub. No.: US 2011/0076331 A1
(43) Pub. Date: Mar. 31, 2011

(54) Use of Deuterium Oxide as an Elastase Inhibitor

(76) Inventor: Thomas Bayerl, London (GB)

(21) Appl. No.: 12/988,694

(22) PCT Filed: Apr. 16, 2009
(86) PCT No.: PCT/IB2009/005999

§ 371 Date: Oct. 20, 2010

(30) Foreign Application Priority Data
Apr. 20, 2008 (EP) 08007676.3

(51) Int. Cl. A61K 33/00; A61K 9/00; A61P 29/00; A61P 9/00; A61P 11/06; A61P 37/08; A61P 11/08; A61P 17/00; A61P 37/06; C12N 9/99

(52) U.S. Cl. 424/484; 424/600; 435/184

(57) ABSTRACT

The invention relates to the use of deuterium oxide (D₂O) as an elastase inhibitor and especially as an inhibitor of the human neutrophil elastase (HNE). The invention also relates to the use of deuterium oxide for the prophylaxis and/or treatment of HNE-related diseases.

The present invention concerns use of deuterium oxide (D2O) as an elastase inhibitor and especially as an inhibitor of human neutrophil elastase (HNE). The invention also concerns use of deuterium oxide to prevent and/or treat HNE-related diseases.

Elastases belong to the family of serine proteases and cleave amides and therefore also amide bonds of peptides and proteins, and esters with absorption of H2O. Human elastases are coded by six different genes and include the human leukocyte elastase (HLE), also known as human neutrophil elastase (HNE) (EC 3.4.21.37). Human neutrophil elastase (HLE) is a glycolized basic serine protease with 218 amino acids with a molecular weight of about 33 kDa. HNE occurs in the azurophil granula of human polymorphonuclear leukocytes (PMN). The intracellular physiological function of HNE consists of degradation of organic foreign particles absorbed by phagocytosis. After activation of polymorphonuclear leukocytes elastase HNE is released from them into the extracellular space (free elastase), with a part remaining bonded to the neutrophil plasma membrane of PMN (membrane elastase). The active extracellular HNE is capable of hydrolyzing almost all extracellular matrix proteins, like elastin, collagen, laminin, cytokines and fibronectin.

Because of their uncontrolled proteolytic activity, however, HNE play a destructive role in a number of pathological processes. These include especially inflammatory diseases, for example, an inflammatory disease of the skin, like neutrophil dermatoses, like palmoplantar pustulosis, subcorneal pustulosis (Sneddon-Wilkinson's disease) (Glinski, W. et al., Basement membrane zone as a target for human neutrophil elastase in psoriasis, Arch Dermatol Res. 1990; 282(8):506-11; Meyer-Hoffert, U. et al., Human leukocyte elastase induces keratinocyte proliferation by epidermal growth factor receptor activation, J Invest Dermatol. 2004 August; 123(2):338-45; Wiedow, O. et al., Lesional elastase activity in psoriasis. Diagnostic and prognostic significance, Arch Dermatol Res. 1995; 287(7):632-5, autoimmune bullous dermatosis, pemphigoid-like bullous pemphigoid, pemphigoid vulgaris, pemphigoid vegetans and pemphigoid foliaceus (Oikarinen, Al et al., Demonstration of collagenase and elastase activities in the blister fluids from bullous skin diseases. Comparison between dermatitis herpetiformis and bullous pemphigoid, J Invest Dermatol. 1983 September; 81(3):261-6; Sliu, Z. et al. A critical role for neutrophil elastase in experimental bullous pemphigoid, J Clin Invest. 2000 January; 105(1):113-23; Shimanovich, I. et al., Granulocyte-derived elastase and gelatinase B are required for dermal-epidermal separation induced by autoantibodies from patients with epidermolysis bullosa acquisita and bullous pemphigoid, J Pathol. 2004 December; 204(5): 519-27), aphthous diseases of the oral mucosa, inflammation of the nasal sinuses, nasal mucosa, middle ear, conjunctiva as well as allergic coryza, asthma, cutaneous vascularitis, pulmonary vascularitis, peritonitis and septic shock (Tsujimoto, H. et al., Neutrophil elastase, MIP-2 and TLR-4 expression during human and experimental sepsis, Shock, 2005 January; 23(1):39-44; Jochum, M. et al., Proteolytic destruction of functional proteins by phagocytes in human peritonitis, Eur J Clin Invest. 1999 March; 29(3):246-55) as well as lung diseases, for example, chronic obstructive pulmonary disease (COPD), cystic fibrosis, chronic bronchitis, pulmonary fibrosis, acute respiratory tract syndrome, pulmonary emphysema (Yoshioka, A. et al. Excessive neutrophil elastase in bronchoalveolar lavage fluid in subclinical emphysema, Am J Respir. Crit. Care Med., December 1995; 152:2127). Hemorrhage (lung damage), etc., cardiovascular diseases, like myocardial infarction, cerebral ischemia as well as other tissue changes after myocardial infarction, cardiac insufficiency and acute coronary syndrome (blood supply disorders of the coronary vessels) (Lechleitner, P. et al. Granulocyte elastase in acute myocardial infarction, Z Kardiol. 1993 October; 82(10):641-7; Dinerman, J. L. et al., Increased neutrophil elastase release in unstable angina pectoris and acute myocardial infarction, J Am Coll Cardiol. 1990 June; 15(7)1559-63; Tiefenbacher, C P. et al., Inhibition of elastase improves myocardial function after repetitive ischemia and myocardial infarction in the rat heart, Pflugers Arch. 1997 March; 433(5):563-70; Bidouard, J P et al., SSR69071, an elastase inhibitor, reduces myocardial infarct size following ischemia-reperfusion injury, Eur J Pharmacol. 2003 Feb. 7; 461(1):49-52; Ohta, K. et al., Elafin-overexpressing mice have improved cardiac function after myocardial infarction, Am J Physiol Heart Circ Physiol. 2004 July; 287(1):H286-92; Shimakura, A. et al., Neutrophil elastase inhibition reduces cerebral ischemic damage in the middle cerebral artery occlusion, Brain Res. 2000 Mar. 6; 858(1):55-60) and allergic diseases like allergies to house dust, mites, plant pollen, allergic asthma, etc. (Sehgal, N. et al., Potential roles in rhinitis for protease and other enzymatic activities of allergens, Gun Allergy Asthma Rep. 2005 May; 5(3):221-6; Gunawan, H. et al., Protease Activity of Allergenic Pollen of Cedar, Cypress, Juniper, Birch and Ragweed, Allergol. Int. 2008 Mar. 1; 57(1):83-91; Runswick, S. et al., Pollen proteolytic enzymes degrade tight junctions, Respirology, 2007 November; 12(6):834-42.

The role of serine proteases overall, like elastases, in pathogenic processes is probably due to an imbalance between the enzyme and its natural endogenous inhibitor. In the case of HNE inhibition of the enzyme occurs by the inhibitor protein alpha1-antitrypsin, A1AT (Stockley, Neutrophils and protease-antiprotease imbalance, Am J. Respir. Crit. Care Med 1999; 160, 49-52). A1AT is normally secreted into the serum from the liver and is present in high excess relative to HNE and irreversibly binds to the active center of HNE and trypsin. In this way the free enzyme HNE is deactivated. However, if an A1AT deficiency is present, this leads to excess of active HNE and uncontrolled activity of the enzyme. The membrane enzyme HNE is also not accessible to A1AT.

In recent years specific serine protease inhibitors have increasingly been sought. Numerous inhibitors for elastases, including HNE, were proposed generally based on small molecules with MW>1 kD, and investigated (Edwards, P. D.; Bernstein, P. R., Synthetic inhibitors of elastase. Med. Res. Rev. 1994, 14, 127-94; J. Potemp et al., The Serpin Superfamily of Proteinase Inhibitors: Structure, Function and Regulation, J. Biol. Chem., Vol. 269, No. 23, Issue of June 10, pp. 15957-15960, 1994).

Such elastase inhibitors can generally be divided into the groups irreversible and reversible inhibitors. While irreversible elastase inhibitors, like HNE inhibitors (including alkyl fluorophosphates, chloromethyl ketones, sulfonyl fluorides) generally enter into a covalent bond with the substrate bonding site of elastases or HNE, reversible elastase or HNE inhibitors are generally characterized by hydrogen bonds, ionic bonds or van der Waals interactions between the inhibitor and elastase or HNE and often have an electrophilic functional group on the C terminus of the P1 residue, which enables them to have higher bonding affinity (including trifluoromethyl ketones, boric acid esters, aldehydes).

Reversible inhibitors are preferred for the development of elastase inhibitors and especially HNE inhibitors for treatment of diseases because of their potentially lower toxic side effects. Nevertheless, these reversible inhibitors still also have significant shortcomings during use in the animal organism. Aldehydes, for example, are subject to the hazard of rapid oxidation to carboxylic acids and racemization in the presence of acids or bases if a chiral center is present on the alpha carbon of the P1 residue. Their low bioavailability, especially their low oral bioavailability because of electrophilic functional groups, is also a factor that limits use in animal organisms, like mammals.

Numerous known HNE inhibitors are peptide-based and consist of no more than 3 to 5 amino acid residues or their equivalent (S. Sinha et al., Conversion of the Alzheimer's α-Amyloid Precursor Protein (APP) Kunitz Domain into a Potent Human Neutrophil Elastase Inhibitor, J. Biol. Chem. Vol. 266, No. 31, Issue of November 5, 21011-21013, 1991). Because of this limited size they can only interact with small areas of the HNE enzyme, which sharply restricts their specificity. When used in the animal organism, as in mammals, such molecules allow us to expect strong side effects. Different sulfonamides, for which a strong inhibiting effect on serine proteases overall has been detected (Sommerhoff, C. P.; Krell, R. D.; Williams, J. L.; Gomes, B. C.; Strimpler, A. M.; Nadel, J. A., Inhibition of human neutrophil elastase by ICI 200,355, Eur. J. Pharmacol. 1991, 193, 153-8; M. Takayama et al., Effects of neutrophil elastase inhibitor (ONO-5046) on lung injury after intestinal ischemia-reperfusion, J Appl Physiol 91:1800-1807, 2001), also have a specificity problem and therefore exhibit side effects when used in the animal organism, like mammals. For larger, also generally peptide-based molecules with an inhibiting effect for HNE there are two additional problems. In the first place, they are subject to the hazard of nonspecific proteolysis by other proteases (for example, by cysteine or metalloproteases) and the products of this reaction can cause side effects (S. Attucci et al., EPI-hNE4, a Proteolysis-Resistant Inhibitor of Human Neutrophil Elastase and Potential Anti-Inflammatory Drug for Treating Cystic Fibrosis, JPET 318:803-809, 2006). Because of their molecule size these higher molecular HNE, inhibitors also could only be used with restriction, since a flexible conformation of the functional group of the inhibitor/substrate that bonds to the catalytic triad of the enzyme is necessary for the specific inhibition of HNE.

The drawbacks of elastase inhibitors known in the prior art and especially HNE inhibitors are therefore a nonspecific effect, undesired side effects, racemization, oxidation in a strongly oxidizing medium, low bioavailability and the hazard of proteolysis by other proteases so that they are inhibited or inactivated in their inhibiting activity.

The task of the present invention is therefore to provide improved inhibitors for elastases and especially improved inhibitors for human neutrophil elastase (HNE).

The task is solved with the present invention. The present invention is based on the finding that deuterium oxide (D2O) is an effective inhibitor for elastases and especially for human neutrophil elastase. Deuterium oxide (D2O), often also referred to as heavy water, is a substance extremely similar to natural water (H2O). D2O and H2O differ physically by substitution of the hydrogen atoms with deuterium atoms. D2O has a roughly 10% higher density and a roughly 25% higher viscosity than H2O. Moreover, the melting and boiling points of D2O are higher than for H2O. A detailed comparison of the properties is provided in the Handbook of Chemistry and Physics, section 6 (Handbook of Chemistry and Physics, David R. Lide, editor, 79^{th} edition, 1998, CRC Press, Boca Raton, USA).

Whereas the physical differences between H2O and D2O are rather limited, there are significant physiological differences (see Kushner, D. J. et al., Pharmacological uses and perspectives of heavy water and denatured compounds, Can J Physiol Pharmacol. 1999 February; 77(2):79-88). For example, it was found that, although many algae and bacteria can exist completely normally over long periods in 100% D2O and this is possible for protozoans at up to 70% D2O, this does not apply for animal cells. At D2O concentrations in the body of more than 20-25% different enzymatically controlled reactions are increasingly altered or inhibited. One cause for this is seen in the altered bonding strength of hydrogen bonds when the hydrogen atom is substituted by a deuterium atom. This generally increased bonding strength occurs both in aqueous solutions of H2O and D2O and also during bonding of water to organic molecules, the effect appearing to be even more strongly pronounced in organic molecules (Cuma, M., Scheiner, S., Influence of Isotopic Substitution on Strength of Hydrogen Bonds of Common Organic Groups, Journal of Physical Organic Chemistry, 1997, Vol. 10, 383-395).

Thus far in the prior art a therapeutic benefit of administering D2O to an animal, especially a mammal, has been considered to be not very effective, since the D2O concentration required for such an effect had to lie above 25% and strong side effects were therefore expected (Kushner, D. J. et al., Pharmacological uses and perspectives of heavy water and denatured compounds, Can J Physiol Pharmacol. 1999 February; 77(2):79-88). Moreover, all published studies in the prior art on D2O effects in cells, organs and organisms come to the conclusion that the effect of D2O lasts only for the duration of D2O administration so that very long cycles of D2O administration would be necessary.

Previous studies from the prior art urgently dealt with the antitumoral and antineoplastic effect of D2O.

For the case of tumor growth in the large intestine and in squamous epithelial cells in the oral cavity and pharynx the antitumor effect of D2O was already experimentally demonstrated in the 1980s on Balb/c/-nu/nu mice (Altermatt, H. J. et al., Heavy water delays growth of human carcinoma in nude mice, Cancer, 1988 Aug. 1; 62(3):462-6). An antitumor effect could also be demonstrated in other tissues. Recently published cell culture studies (Hartmann, J. et al., Effects of heavy water (D2O) on human pancreatic tumor cells, Anticancer Res. 2005 September-October; 25(5):3407-11) with three tumor cell lines from the pancreas also show an antitumor effect. Other studies found clear results for D2O-mediated antitumor effect in tissues of neoplastic brain cells (Uemura. T. et al., Experimental validation of deuterium oxide-mediated antitumoral activity as it relates to apoptosis in murine malignant astrocytoma cells, Neurosurg. 2002 May; 96(5):900-8) and suggest that D2O can induce apoptosis in malignant astrocytoma cells. In this study the authors also concluded that D2O has a cytotoxic effect on tumor tissue and therefore represents a cytostatic.

Published studies in which an established cytostatic was administered together with D2O instead of H2O to treat murine, xenotransplanted tumors (Altermatt, H. J., Heavy water (D2O) inhibits growth of human xenotransplanted oropharyngeal cancers, An animal experiment study in nude mice, Laryngol Rhinol Otol (Stuttg), 1987 April; 66(4):191-4) confirm an additional antineoplastic effect of D2O.

However, nowhere in the prior art has D2O thus far been described as an effective inhibitor of enzymatic activities, like an elastase inhibitor and especially as an inhibitor of human neutrophil elastase (HNE).

The effect mechanism of D2O according to the invention as an elastase inhibitor, especially as an HNE inhibitor, is generally based on changes in hydrogen bond energy, when hydrogen atoms of such bonds are replaced by deuterium atoms. For proteins and enzymes this is significant in a number of respects. Hydrogen bonds (H bonds) stabilize their tertiary and quaternary structure and therefore influence the spatial arrangement of individual areas (domains) relative to each other and their (functional) changes by convolutions.

The tertiary structure of a protein is understood to mean the spatial structure of proteins higher than the secondary structure (amino acid chain), i.e., the complete three-dimensional structure of the amino acid chain, which is essential for biological function of the protein. During convolution of the amino acid chain to a three-dimensional structure of the protein the hydrophobic areas are arranged in the interior of the protein, whereas the hydrophilic areas point outward and therefore face the aqueous surroundings of the protein. Stabilization of the tertiary structure of a protein occurs via different bonds: disulfide bridges, ionic bonds, hydrogen bonds and hydrophobic interactions.

The quaternary structure consists of the fusion of several protein molecules into a functional complex. This fusion occurs via non-covalent interactions: hydrogen bonds between peptide bonds and side chains, ionic bonds and van der Waals forces. The fusion of subunits in hydrophobic areas, i.e., areas in the interior of the protein, primarily occurs via the hydrophobic effect.

Through changes in hydrogen bond energies and therefore stabilization of the protein or enzyme by H bonds of the tertiary and quaternary structure the elastase, especially HNE, can enter into a conformation that sterically hinders the substrate bonding or makes it energetically unfavorable. The result is partial or complete inhibition of its activity.

H bonds in many cases also participate in the bonding of substrates and their modification by enzymes. A change in bond energy can have a variety of effects here, which as a result can produce a change in reaction pathway and/or reaction rate. As mentioned, elastases belong to the family of serine proteases and are characterized by a specificity for branched-chain aliphatic groups on P1 (substrate). The catalytic triad of the enzyme HNE consists of Ser 195, His 57 and Asp 102 residues (chymotrypsin numbering, see Greer J, Comparative modeling methods: Application to the family of the mammalian serine proteases. Proteins Struct Funct Genet, 1990, 7:317-334 for nomenclature) and an oxyanion “hole” in the substrate absorption area. The substrate bonds to form a Michaelis complex in which the carbonyl group of the amide bond being cut is exposed to the hydroxyl group of Ser 195 and is subject to basic catalysis by the imidazole side chain of His 57. The resulting tetrahedral intermediate is stabilized by hydrogen bonds, which bond to the NH framework of Ser 195 and Gly 193. Water addition to the complex then occurs so that a second tetrahedral intermediate is formed and finally is broken down by acid-supported catalysis via His 57 with regeneration of Ser 195 and the N-terminal fragment of the cut off substrate. Hydrogen is involved in this catalytic process in three essential steps. Initially during formation of an H bond between Ser 195 and His 57, which only occurs during substrate bonding and which is critical for the catalytic effectiveness of the triad (Perona, J. J. and C. S. Craik, Structural basis of substrate specificity in the serine proteases, Protein Science (1995), 4:337-360). Also during formation of H bonds for intermediate stabilization and finally during catalytic water addition to the complex. Since deuterium bonds (D bonds) have a significantly different bond energy in comparison to H bonds (the additional neutron in deuterium restricts some high-frequency degrees of freedom in the petahertz (10¹⁵ Hz) frequency range so that D bonds have slightly reduced bond spacings among other things), the catalytic effect of His 57 and bonding via D bonds to Ser 195 and Gly 193 is disturbed. Catalytic conversion is slowed or completely inhibited.

Another effect mechanism involves the altered bond spacings and bond energies from hydrogen-deuterium substitution in the H bonds necessary for substrate bonding. Because of this, specific bonding of substrates is either hampered or suppressed. On the other hand, certain substrates can be bonded so firmly to the enzyme that they block the catalytic triad and inhibit catalytic activity in the enzyme on this account.

A third effect mechanism is the substitution of hydrogen in the H bonds between His 57 and Asp 102 by a deuteron. The change in bond energy and bond spacing effectively leads to rupture of this bond in the catalytic triad. The results of such a bond rupture have been thoroughly investigated by molecular dynamic simulations (MD) and clearly show that through a bond rupture the catalytically productive conformation of the triad is lost (E. Lau and T. C. Bruice, Consequences of Breaking the Asp-His Hydrogen Bond of the Catalytic Triad: Effects on the Structure and Dynamics of the Serine Esterase Cutinase, Biophysical Journal, Vol. 77, 1999, 85-98).

The high specificity of deuterium oxide as elastase inhibitor according to the invention, which was demonstrated above in detail for HNE, is achieved on the one hand by the individually listed effect mechanisms, but on the other hand, mostly by the reinforcing interaction of all mechanisms. HNE differs from the trypsin, metallo- and chymotrypsin proteases with respect to substrate specificity in that for HNE this is not additionally determined by bond motifs at a distance of S1 (i.e., S2-S4). Much greater significance for specific substrate bonding than in trypsin, chymotrypsin and metalloproteases is therefore attached to the H bonds and their changes from deuterium substitution in the region of S1 of HNE. In this way D2O can specifically inhibit the effect of HNE. The effect mechanism described for HNE can also be transferred to other elastases.

According to the invention, by administering D2O a situation is achieved in which the bond energies and bond spacings of the stabilizing hydrogen bonds of elastases, mostly HNE, are altered by substitution of hydrogen atoms with deuterium atoms. Through this higher bonding strength the conformation of the enzyme is altered, which leads to its partial or complete inactivation of catalytic activity. In other words, the enzyme activity of HNE is fully or partially inhibited. The specificity for elastases, or for HNE, is achieved by the described characteristic triad (Ser 195/His 57/Asp 102) and the conformation change of the enzyme (by substitution of hydrogen atoms with deuterium atoms).

D2O according to the invention is therefore an effective and specific elastase inhibitor, especially an inhibitor of HNE and therefore effective in the prevention and/or treatment of diseases that are associated with nonspecific or uncontrolled elastase activity, mostly the activity of human neutrophil elastase.

The present invention in its first two variants concerns the use of deuterium oxide (D2O) as an elastase inhibitor and as an inhibitor of human neutrophil elastase (HNE).

In another variant the present invention concerns the use of deuterium oxide as an elastase inhibitor and especially as an inhibitor of human neutrophil elastase (HNE) for prevention and/or treatment of HNE-related diseases.

In another variant the present invention concerns the use of deuterium oxide as an elastase inhibitor and especially as inhibitor of human neutrophil elastase (HNE) to produce a drug for prevention of and/or treatment of HNE-related diseases.

The term “use according to the invention” subsequently includes the use of D2O as an elastase inhibitor and especially as an HNE inhibitor and the use of D2O as an elastase inhibitor and especially as an HNE inhibitor to prevent and/or treat HNE-related diseases.

The terms “prevention and/or treatment” according to the invention refer to any measure appropriate for treatment of an elastase or HNE-related disease, which represents either a preventive treatment of such a disease or its symptom or the avoidance of the occurrence of such a disease, for example, after a treatment time has been completed (prevention) or represents the treatment of symptoms of an already developed disease (therapy).

“Elastase-related diseases” or “HNE-related diseases” are according to the invention understood to mean a pathological picture characterized by uncontrolled or unspecific enzymatic reaction or activity of an elastase or human neutrophil elastase (HNE) in which the reaction or activity of the enzyme especially occurs to a high degree.

An “inhibitor” according to the invention refers to a substance that inhibits the enzymatic reaction or activity of an elastase and especially of a human neutrophil elastase (HNE) or delays it. Inhibition or obstruction of HNE is preferably reversible. The terms “substance”, “compound”, “molecule” and “agent” are used synonymously according to the invention.

A preferred variant of the present invention consequently concerns the use of deuterium oxide according to the invention, in which the activity of elastase and especially human neutrophil elastase (HNE) is impeded or inhibited.

The term “obstruct” or “obstruction” according to the invention is to be understood to mean that the enzymatic activity of elastase and especially human neutrophil elastase (HNE) is slowed (delayed) and/or reduced, preferably up to about 5%, more preferably up to about 10% and especially up to about 20% and more preferably up to about 30% and also more preferably up to about 40% and even more preferably up to about 50% and most preferably up to about 60% relative to the enzymatic activity of elastase and especially HNE without administration of D2O.

The term “inhibit” or “inhibition” according to the invention is to be understood to mean that the enzymatic activity of elastase and especially human neutrophil elastase (HNE) is slowed (delayed) and/or reduced, preferably up to about 50%, more preferably up to about 60% and especially up to about 65% and more preferably up to about 70% and also more preferably up to about 80% and even more preferably up to about 90%, even more strongly preferably up to about 95%, more strongly preferably up to about 98%, and most preferably up to 100% relative to the enzymatic activity of elastase and especially HNE without administration of D2O.

A preferred variant of the present invention concerns use of D2O for prevention and/or treatment of elastase-related and especially HNE-related diseases in which inflammatory disease, lung disease, heart disease and/or cardiovascular diseases and/or allergic diseases are involved.

The inflammatory diseases according to the present invention are preferably inflammation of the skin, nasal mucosa, oral mucosa, especially aphthous diseases of the oral mucosa, conjunctiva, nasal sinuses, or allergic coryza, asthma, cutaneous vascularitis, pulmonary vascularitis, peritonitis or septic shock.

The inflammatory diseases according to the present invention are preferably inflammation of the skin, selected from the group consisting of neutrophil dermatoses, like palmoplantar pustulosis, subcorneal pustulosis (Sneddon-Wilkinson's disease), autoimmune bullous dermatosis, pemphigoid, like bullous pemphigoid, pemphigoid vulgaris, pemphigoid vegetans and pemphigoid foliaceus.

The lung diseases according to the present invention are preferably a lung disease selected from the group consisting of chronic obstructive pulmonary disease (COPD), cystic fibrosis, chronic bronchitis, pulmonary fibrosis, acute respiratory tract syndrome, pulmonary emphysema and hemorrhage.

The heart diseases and cardiovascular diseases according to the present invention are preferably a heart disease and/or a cardiovascular disease selected from the group consisting of myocardial infarction, cerebral ischemia, cardiac insufficiency and acute coronary syndrome.

The allergic diseases according to the present invention are preferably an allergic disease selected from the group consisting of house dust allergy, mite allergy, plant pollen allergy and allergic asthma.

Effective prevention and/or treatment of elastase and especially HNE-related diseases can be achieved in particular by administering a pharmaceutical agent, more precisely an inhibitor, which, when taking into consideration the type of administration, has preferably all of the following properties:
a) in elastase inhibitors and especially HNE inhibitors to be administered topically: local applicability by topical administration over any long period of time and with high percutaneous transport kinetics of the agent (the same applies for parenteral administration);
b) in elastase inhibitors and especially HNE inhibitors to be administered via an aerosol: transportability into the lungs or nose in any desired particle size for random or selective control of the agent (elastase or HNE inhibitor) within the lung tissue;
c) a large homogeneous agent distribution in the area of the effect location and avoidance of local overconcentrations;
d) preferred enrichment of the elastase inhibitor and especially the HNE inhibitor in the affected area of the body, if possible connected with retardation of transport into the circulation, i.e., into blood vessels;
e) an inhibiting effect, i.e., an inhibiting effect on the activity of elastase and especially HNE in the affected body area;
f) essential tolerance of the elastase inhibitor and especially HNE inhibitor in the surrounding nonpathologically affected tissue and circulation to avoid side effects, especially with respect to immune reactions.

D2O as agent and elastase inhibitor and especially as HNE inhibitor has significant advantages relative to known elastase inhibitors and especially HNE inhibitors for treatment of elastase or HNE-related diseases mostly because of its following properties:
1) through the possibility of administering D2O topically on the skin a high concentration sufficient for therapeutic efficacy (of more than 20% in terms of the total water content of the cell) of D2O can be achieved in the epidermis or dermis of the skin because of the high cutaneous, percutaneous and intracutaneous transport of D2O in the skin, without exposing other organs of the body to similarly high concentrations of D2O, as can occur during systemic administration. Therefore a critical problem discussed in the prior art for achieving therapeutically effective D2O concentrations at the effect location (more than 20% D2O in terms of to the total water content of the cell) without strong side effects in other organs or healthy surrounding skin tissue is solved. The basis for this is directed transport of D2O through the stratum corneum of the skin to the epidermis or dermis;
2) The state of aggregation of D2O during topical administration can be liquid, gaseous or solid. Transport in the skin can occur through direct contact of D2O or a D2O-containing formulation with the skin and indirectly by diffusion through an intermediate layer (for example, air, porous membrane, polymer network);
3) For administration of pure liquid D2O alone (pure D2O), it should be stated that D2O has a unique advantage compared to all other liquid pharmaceutical agents. Like normal water (H2O), it can be transported in the skin and the penetration depth of D2O into the skin can also be adapted to the therapeutic objective through the strength and direction of the osmotic gradient and manipulation of these two quantities;
4) It should be stated that for administration of D2O as aerosol the surface tension of D2O is almost identical to that of H2O and permits generation of D2O aerosols with deliberately controllable particle sizes in the range from 0.1 to 10 μm. For this purpose, for example, but not exclusively, established methods of mechanical atomization can be used. Through a deliberate choice of particle size D2O aerosols can be directed into almost any area of the lungs by inhalation. There they would initially be enriched in alveoli and then transported as molecule clusters or D2O individual molecules into the lung tissue through the barriers. By size selection of the D2O particles the deliberate introduction (optimization of the effect location) of the agent D2O is therefore possible for specific lung areas.
5) For administration of D2O as aerosol, it should also be stated that D2O aerosol has a unique advantage compared to all other liquid aerosolizable pharmaceutical agents. Whereas in all other cases excipients must be added in order to transport the pharmaceutical agent stably into the lungs via the aerosol, this is not necessary in D2O, since it is already optimally aerosolizable as a pure molecule without additives. The efficacy in demixings that adversely affect the lungs, as can occur in other substance mixtures (agent/excipient) during aerosolization and the side effects caused by excipients can therefore be ruled out. “Aerosolizable” or “aerosolizability” is to be understood to mean the essential possibility of converting a substance to an aerosol with controlled particle size by means of methods that are state of the art.

According to the invention not only is the effect of D2O demonstrated as elastase inhibitor and especially HNE inhibitor, but also the administration of a combination of D2O with another pharmaceutical agent, preferably another protease inhibitor, preferably a serine protease inhibitor, can further intensify this effect. In addition to or instead of an additional pharmaceutical agent, the use of D2O together with another non-pharmaceutical agent can also occur.

Another preferred variant concerns the use of deuterium oxide according to the invention in which deuterium oxide is used in combination with at least one additional pharmaceutical agent and/or at least one additional non-pharmaceutical agent. Such a combination of D2O and at least one additional pharmaceutical agent and/or at least one additional non-pharmaceutical agent is referred to below as “combination according to the invention”.

All uses and administrations or routes of administration of D2O according to the invention disclosed in this description are also applicable without restriction to a combination according to the invention unless otherwise stated.

The term “pharmaceutical” agent according to the present invention denotes any inorganic or organic substance to which a pharmacological effect is attributed. D2O and other elastase and especially HNE inhibitors are also considered pharmaceutical agents according to the present invention.

The term “non-pharmaceutical” agent according to the present invention denotes any pharmacologically compatible and therapeutically useful substance that is not a pharmaceutical agent but can be formulated together with the pharmaceutical agent in the pharmaceutical composition in order to influence the qualitative properties of the pharmaceutical composition, especially to improve them. Preferably the non-pharmaceutical agents have no noticeable or at least no undesired pharmacological effect with respect to the intended therapy.

The strategic objectives of these mixtures of additional pharmaceutical and/or non-pharmaceutical agents lie in intensifying the HNE-inhibiting effect and/or improving the tolerability of D2O.

The concentration of additional pharmaceutical agents used in addition to D2O as a pharmaceutical agent according to the invention referred to the total solution of a combination according to the invention, lies in the range from at least 10⁻⁸M to at least 5·10−2M, preferably at least 10−7M to 10−3M, most preferably from at least 10−6M to at least 10−2M. A particularly preferred concentration range lies in the range from at least 10−9M to at least 10−2M.

Preferred additional pharmaceutical and non-pharmaceutical agents according to the invention and their effect are given below, the present invention not being restricted to them:

Appropriate pharmaceutical agents are especially: sulfonamides, antibiotics (especially penicillin), corticoids, alkyl fluorophosphates, chloromethyl ketones, sulfonyl fluorides, trifluoromethyl ketones, boric acid esters, aldehydes, short-chain peptides (especially peptides with less than 10 amino acids), cytostatics, chemotherapeutics, synthetic and plant agents with inflammation-inhibiting effect.

Appropriate non-pharmaceutical agents include pharmaceutically compatible inorganic or organic acids or bases, polymers, copolymers, block copolymers, simple sugars, multiple sugars, ionic and nonionic surfactants or lipids, pharmacologically safe salts, for example, sodium chloride, flavorings, vitamins, for example, vitamin A or vitamin E, tocopherols or similar vitamins or provitamins occurring in the human body, antioxidants, like ascorbic acid, as well as stabilizers and/or preservatives for lengthening the use and storage time of a pharmaceutical agent or formulation and other ordinary non-pharmaceutical agents or excipients and additives known in the prior art, as well as their mixtures. Additional preferred non-pharmaceutical agents according to the invention are especially all substances capable of forming aqueous gels, like natural and synthetic water-soluble polymers, which can form networks.

Preferred other (additional) non-pharmaceutical agents and their effect are mentioned according to the invention, the present invention not being restricted to them:

Water-Soluble Excipients and Additives
By addition of water-soluble excipients and additives, like pharmaceutically compatible inorganic or organic acids, bases, salts and/or buffers to adjust the pH value, the physiological compatibility of D2O in the lungs can be improved. Examples of preferred inorganic acids are hydrochloric acid, hydrobromic acid, nitric acid, sulfuric acid and phosphoric acid, hydrochloric acid and sulfuric acid being particularly preferred. Examples of particularly suitable organic acids are malic acid, tartaric acid, maleic acid, succinic acid, acetic acid, formic acid and propionic acid and especially ascorbic acid, fumaric acid and citric acid. Mixtures of the mentioned acids can optionally also be used, especially acids that have other properties in addition to their acid properties, for example, when used as flavorings or antioxidants, like citric acid or ascorbic acid. Examples of pharmaceutically-compatible bases are alkali hydroxides, alkali carbonates and alkali ions, preferably sodium. Mixtures of these substances can be used in particular to adjust and buffer the pH value, of particular preference here are potassium hydrogen phosphate and dipotassium hydrogen phosphate as well as sodium hydrogen phosphate and disodium hydrogen phosphate. Preferred buffers according to the invention are also PBS, HEPES, TRIS, MOPS as well as other physiologically compatible substances with a pK value in the range 5.0 to 9.0.
The concentration of these substances, referring to the total solution, preferably lies in the range from micromolar to millimolar, especially in the range 1-100 mM.

Water-Soluble Non-Cytotoxic Molecules
By adding water-soluble non-cytotoxic molecules, like certain polymers (for example, not restricted to these, dextran, polyethylene glycol, cellulose, hyaluronic acid), copolymers and block copolymers the additional delay (retardation) of transfer of D2O into the blood stream can be achieved due to their high water bonding capacity. In the case of using D2O as an aerosol according to the invention, the stability of the D2O particles and their transport into the alveoli of the lungs can be altered and improved or optimized by the capability of polymers that alter the density and/or viscosity of D2O.
The concentration of these substances, in terms of total solution, lies in the range from millimolar to molar, preferably in the range 1-500 mM.

Water-Soluble Non-Polymer Molecules
By adding water-soluble non-polymer molecules, which alter the density and/or viscosity of D2O, for example, but not restricted to these, simple sugars and multiple sugars, especially glucose, sucrose, dextrose, maltose, starch and cellulose, transport of D2O can be altered and improved or optimized—and in the case of using of D2O as an aerosol according to the invention the stability of the D2O particles in the alveoli of the lungs could be changed and improve or optimized.
The concentration of these substances referred to the total solution preferably lies in the range from millimolar to molar with particular preference in the range from 1.0 mM to 1.5M.

D2O Interfacial Tension-Altering Substances
By adding substances that alter interfacial tension of D2O, for example, but not restricted to them, ionic and nonionic surfactants or lipids, especially a mixture of surfactants and lipids, which are referred to as lung surfactants in the literature, the physical-chemical state, especially the aggregation capability of D2O particles, for example, aerosol can be altered. This can be used, for example, in the case of use of D2O as aerosol according to the invention to alter the stability and transport of the particles into lung tissue. Moreover, such molecules in combination with pharmaceutical agents poorly soluble in D2O can assume the function of a solubilizer and therefore permit, among other things (but not restricted to this), the transport of higher amounts of pharmaceutical agent per dose, for example, per aerosol particle.
The concentration of these substances in terms of the total solution preferably lies in the range from micromolar to millimolar, with particular preference in the range 1-500 mM.

D2O used according to the invention is preferably present as liquid. D2O is preferably present in the solution, preferably with H2O (water) as solvent and is referred to also as “D2O/H2O solution”, when H2O is contained, or as “D2O solution” or “pure D2O”, when no H2O is contained. Pure D2O contains D2O preferably in a concentration range from 98.1 to 100%, preferably 98.5 to 99.9% with particular preference of 99.7% in terms of the total water content of the solution. A D2O/H2O solution according to the invention containing D2O preferably in a concentration range from 1 to 98%, preferably 5 to 95%, also preferably 10 to 90%, also preferably 15 to 80%, more preferably 20 to 70% and even more preferably 30 to 60% and most preferably 40 to 50% in which these data refer to the total water content of the mixture of D2O and H2O.

Preparation of a D2O/H2O solution according to the invention, D2O solution and a combination similar to this according to the invention occurs, for example, by mixing of the components, especially D2O, optionally H2O and optionally at least one other pharmaceutical and/or non-pharmaceutical agent. A solution, as described subsequently can also be added by mixing. Mixing of H2O and at least one additional pharmaceutical and/or non-pharmaceutical agent or solvent to D2O preferably occurs in the liquid state of aggregation. Preparation, however, can also be achieved by any appropriate method.

All applications and administrations of D2O according to the invention disclosed in this description are applicable without restriction to both D2O/H2O solutions and D2O solutions if nothing to the contrary is indicated. Applications of D2O/H2O solutions and D2O solutions according to the invention also find use in the combinations, layer systems, patches and bandages according to the invention, formulations and aerosols with unrestricted use, if nothing contrary is indicated.

Use of D2O according to the invention, as further described below, can also occur as aerosol, vapor or formulation, especially as cream, ointment, gel or hydrogel.

In a preferred variant at least one additional pharmaceutical agent or additional non-pharmaceutical agent is bonded to D2O. “Bonded” according to the present invention means that the pharmaceutical or non-pharmaceutical agent is hydrated by the D2O.

In other preferred variants the D2O or combination according to the invention is contained in an appropriate solvent. A solvent according to the invention can be an inorganic or organic solvent. Appropriate solvents of the present invention should preferably be physiologically compatible with the organisms (especially mammal) to which the agent with solvent is administered, i.e., trigger no side effects, for example, toxic side effects. A particularly preferred solvent is distilled water. Ethanol/water mixtures are also preferred; the weight percent of ethanol in these mixtures is preferably in the range between 5% and 99% ethanol, also preferably in the range from 10% to 96% ethanol, more preferably between 50% and 92% and most preferably between 69% and 91% ethanol.

Administration of D2O can occur according to the invention topically, transdermally, nasally, rectally, parenterally, via a perfusion, via an endoscope or as aerosol or dry powder formulation.

Topical and transdermal administrations occur by applying D2O to the skin, preferably as D2O-containing liquid (D2O solution, D2O/H2O solution), gas (aerosol or vapor), formulation, preferably as a ointment, cream, lotion or emulsion or as D2O-containing gel or hydrogel. These forms of administration can preferably occur via a patch or bandage.

Nasal administration preferably occurs via a D2O-containing powder or D2O-containing liquid formulation which are trickled or snorted into the nose.

Rectal administration preferably occurs via a D2O-containing suppository or via injection of a D2O-containing liquid formulation.

Parenteral administration preferably occurs as injection or infusion of a D2O formulation and includes, for example, intravenous, intra-articular, intra-arterial, intralymphatic, subcutaneous, intracutaneous, intrapulmonary, intraperitoneal, intracardial, intrathecal, intrapleural, intravitreal administration.

Administration via a perfusion occurs according to the invention preferably in heart and/or cardiovascular diseases.

Administration via an endoscope occurs according to the invention preferably in lung diseases.

Administration can also occur by inhalation, for example, as an aerosol or endobronchially (via a tube).

A preferred topical or transdermal administration of D2O according to the invention is particularly advantageous in the described inflammatory diseases of the skin and in allergic diseases. In the preferred topical or transdermal administration according to the invention locally high, therapeutically effective D2O concentrations can be used on the skin and the burdens on the system (i.e., the circulation) and the side effects on healthy skin tissue not being treated as well as the tissue of other organs (for example, liver and kidneys, which could be caused by high concentration of D2O of more than 20% D2O in terms of the total water content) can be simultaneously reduced or completely avoided.

The transport of D2O from the skin cells into the system can also be prevented or restricted by means well known in the prior art. Examples of these means include deliberate manipulation of the osmotic gradient through the skin (i.e., between the systemic part and the skin surface) by reducing the water potential of the topically applied D2O by means of substances that are appropriate for altering this water potential, especially physiologically compatible salts, like sodium chloride, water-soluble polymers and other non-pharmaceutical substances.

Topical administration of D2O can also occur via a patch or bandage. The use of D2O according to the invention is particularly preferred according to the invention in which D2O is topically applied with or via a patch or bandage.

“Patches” or “bandages” according to the invention are to be understood to mean all devices that can be fastened to the skin by mechanical or chemical interaction, physisorption, adhesion or other physical-chemical processes, which are suitable for covering a selected skin area occlusively or non-occlusively for a long period appropriate for the intended treatment and permitting and/or supporting the supply of D2O to the skin. Patches and bandages applicable according to the invention as application systems for local release of agents on the skin (for example, heat bandages) and for controlled systemic release of agents (for example, opiate depot patches, nitroglycerine depot patches) are known in the prior art. “Depot patches” or “depot bandages” are additionally to be understood to include the capability of the patch or bandage to store D2O and its controlled released to the skin over a period of days or weeks, in addition to the properties described above. Such depot patches or depot bandages are included subsequently under the terms patch or bandage.

Overall the following problem must be kept in mind for each controlled release of D2O to the skin: release from a liquid applied directly to the skin or formulation as a ointment, cream or gel can be hampered by the direction of the osmotic gradient within the skin from the inside out, since D2O in the liquid, ointment, cream or gel under some circumstances has a lower water potential than H2O in the skin and in the underlying vessels.

A particularly preferred variant of topical application of D2O is therefore preferred to regulate the depth or degree of penetration of D2O into the skin by deliberate manipulation of the osmotic conditions in the skin area being treated and therefore control it preferably up to the epidermis or dermis. This can be achieved by the selected composition of an applied combination according to the invention, in which substances are added that are capable of altering the osmotic conditions on the surface of the skin.

Another possibility for controlled penetration of D2O into the skin consists of using one or more membranes or films that permit passage of water and gases, but prevent larger molecules or particles (including bacteria, viruses, individual cells). Examples of such membranes and films usable according to the invention are known in the prior art and have numerous applications, for example, in textiles under the trade names GORE-TEX® or in so-called biofilms or breathable patches like Tegaderm® in medicine.

Application of D2O according to the invention is therefore particularly preferred when D2O is applied topically with a patch or bandage in which the patch or bandage is used in combination with at least one membrane or at least one film. The membrane of which there should be at least one or film of which there should be at least one is preferably a micro- or nanoporous membrane or film.

An example of a preferred arrangement for such a topical application of D2O according to the invention consists of the following components:
a previously described micro- or nanoporous membrane or film, which is applied directly to the skin,
followed by a D2O layer containing the D2O,
optionally followed by a so-called occlusion layer, which prevents or regulates evaporation of D2O outward and at the same time represents a mechanical protection that prevents escape of D2O as liquid and
followed by a patch or bandage.

Another example of an arrangement for these sorts of topical applications of D2O according to the invention consists of the following components:
a previously described micro- or nanoporous membrane or film, which is applied directly to the skin,
followed by a D2O layer which contains the D2O,
optionally followed by a so-called occlusion layer, which prevents or regulates evaporation of D2O outward and at the same time represents a mechanical protection that prevents escape of D2O as liquid and
followed by an additional already mentioned micro- or nanoporous membrane or film.

If required, additional D2O layers can naturally be added, which contain D2O and which are separated by membranes or films so that the depot effect or transfer of D2O to the skin can be altered, for example, the release amount and/or time of D2O. The employed term “D2O layer” pertains to a liquid, a D2O solution (pure D2O), D2O/H2O solution, a combination according to the invention and a formulation according to the invention of D2O, especially as a cream, ointment, gel or hydrogel.

Layers can also preferably be added, which have chemical, electrical or thermal properties suitable for manipulating the transfer of D2O into the skin and/or the time of its release. Examples of this include layers that are appropriate for building up and/or maintaining an electrical, thermoelectrical, thermal or chemical potential (or a combination thereof) over the underlying layers and the skin. This can be achieved, for example, by electrodes either embedded in the described membranes or films or situated on them, which are supplied from the outside with a current (dc, ac or high-frequency currents) or which generate electrochemical potentials by deliberate choice of the electrode material with the D2O layer as electrolyte.

All of the previously described D2O layers, occlusion layers, layers with chemical, electrical or thermal properties, membranes and films in any number, combination and arrangement suitable for the application are referred to subsequently as “layer system”. Such a layer system is preferably used in conjunction with one of the previously mentioned (depot) patches or (depot) bandages.

By variation of the morphology (pore size, membrane or film thickness, surface roughness and surface profile) and surface properties (for example, hydrophilic or hydrophobic, chemical coatings, covalently bonded or adhesively bonded, functional groups, bonding or incorporation of inorganic or organic substances) of the membrane or film in direct contact with the skin, the transfer of D2O from a described patch, bandage or layer system into the skin can be deliberately influenced or altered.

Another variation of entry of D2O to the skin is possible by deliberate use of adhesives, which can be used for mechanical fastening of the (depot) patch or (depot) bandage on the skin, but are not absolutely necessary. The adhesives generally used for topical applications of patches and bandages have a rather hydrophobic character, which can prevent passage of D2O through the adhesive layer. By mixing additives into the adhesive preparation a change in these properties can be achieved. Organic and/or inorganic substances and compounds that are capable of altering the permeation properties of D2O through the adhesive layer are considered as such “additives”. Examples of such substances include polymers, copolymers, block polymers, block copolymers, surfactants, peptides, proteins, nucleic acids, sterols and steroids.

Another preferred variant use of D2O according to the invention occurs preferably with a patch or bandage, topically on the skin via an arrangement that permits transfer of D2O into the skin largely or exclusively via the vapor phase. This means D2O used as liquid according to the invention evaporates as molecular D2O and comes in contact with the skin as vapor. The concentrations of D2O therefore correspond to the concentrations of a D2O-containing liquid described above. Gaseous D2O has the advantage of particularly easy penetration into the skin. To produce this evaporation thermal energy is required, which can be obtained either from the skin itself or from an external heat source, for example, during use of a described patch or bandage or the layer system described above with electric heating incorporated in it (for example, Peltier heating). In addition to this thermal energy, by deliberate modifications, for example, the choice of morphology (especially pore size and surface coating) of a first membrane or film described above situated on the skin of the layer system, a situation can be achieved in which only gaseous D2O can penetrate through the membrane and film to the skin while liquid D2O is retained. In this variant the amount and duration of the release of D2O via the vapor phase can also be controlled and/or altered by the aforementioned changes in the layer system and corresponding modifications to any additionally employed membranes or films.

A preferred administration of D2O as aerosol according to the invention is especially advantageous in the described lung diseases, inflammation of the nasal sinuses, nasal mucosa, allergic coryza, asthma and allergic asthma and the other allergic diseases.

A preferred administration of D2O as aerosol according to the invention occurs via inhalation of D2O. The inhalation of agents through the lungs of an organism, for example, a mammal, is a known and increasingly employed technique for years for local and systemic release of these substances. It is based on the transport of particles of the size from a few hundred μm to a few nm in the air stream during inhalation, followed by deposition of the particles in the alveoli, from which they can then penetrate into the system and be transported to the effect location within the body. In many cases the lungs themselves are the effect location. All molecules or macromolecules lying in the size range from 0.005 μm to 100 μm are referred to as particles according to the invention subsequently. Structures denote molecules or macromolecules, regardless of whether they have a solid or liquid state of aggregation. The particles are formulated as an aerosol for this purpose and inhaled by the patient, preferably via appropriate inhalers (also called atomizers).

Another object of the present invention is therefore a D2O-containing aerosol.

An aerosol is understood to mean solid or liquid suspended particle with a diameter of about 0.0001 μm to about 100 μm, in gases, especially air, in which the composition and form of the aerosols can vary very sharply. Aerosols can be prepared artificially by dispersion and condensation methods well-known in the prior art. They can be used without a propellant or used in combination with a liquid compressed gas as a propellant in spray cans. Aerosols (with and without propellant) are often used in medicine for so-called aerosol therapies for the transport of pharmaceutical agents into the lungs. If the term aerosol is used below, it refers to medical aerosols. The smallest pharmaceutically active particles in aerosols are nucleic acids, peptides or proteins, the largest particles are mist particles. Aerosols often consist of mixtures of particles of different particle sizes and in so doing embody a polydispersed size distribution.

The preparation of aerosols according to the invention (with and without solvents) can occur through a method known in the prior art. Numerous standard methods are known for this purpose.

The inhalation of aerosols occurs orally and nasally by the organism being treated, especially a mammal and goat, preferably via an inhaler. After inhalation of particles into the lungs a certain fraction of the particles deviates from the flow line of the aerosol and then enters into contact with the moist surface of the air spaces. This phenomenon is generally referred to as particle deposition or deposition and is subject to three physical mechanisms:
Impaction, due to the inertia of the aerosol, occurs in particles with a diameter from about 3 μm,
Sedimentation, due to the weight of the particles, occurs in particles with a diameter from about 0.5 to 1 μm and
Diffusion, due to Brownian molecular motion, occurs in particles with a diameter of less than about 1 μm.

Choice of a particle size of the pharmaceutical agents to be transported is therefore a critical factor for the deposition mechanism in the lungs.

Recent developments of inhalation technology can be divided into two areas of emphasis:
1) Optimization of release methods in the sense of control via the physical properties of the particles being inhaled and
2) Optimization of the agent formulations used for release in terms of improvement of colloidal properties of the particles for inhalation and their systemic efficacy.

All developments of atomization or inhalation systems for solids, liquids and gases can be combined under point 1). They are concentrated in the fields of mechanical atomization, evaporation, etc. with the objective of controlling especially the particle size and particle stability. The developments under 2) include improvements of agglomeration behavior of the particles, their release and transport properties in the alveoli and the stability of the agents in the particles.

Since the physical-chemical properties of the particles (especially size, stability, solubility in an aqueous environment, surface charge, bonding properties, nucleation behavior, interface properties) are determined by the pharmaceutical agent itself, individual adaptation of the release methods is often necessary for special pharmaceutical agents and their formulations. The result of such an often lengthy adaptation process (in view of the number of parameters to be considered) is almost always a compromise between (i) the inhalation properties, (ii) the transport properties of the particles in the lungs or in alveoli, (iii) the individual inhalation technique of the patient and (iv) the therapeutic efficacy in the lungs or in the system.

Overall this can summarized as follows:
1) Gaseous substances are ideally suited for application via the lungs (for example, narcosis gases during artificial ventilation), since they generally pass through lung barriers without problem and are systemically available almost immediately. However, they are also quickly eliminated from the system again and therefore have no depot effect and do not permit optimization of the effect location in the system. “Depot effect” is understood to mean the time-delayed release of the pharmaceutical agent in the system and (with optimization of the effect location) the above-average enrichment of the pharmaceutical agent in the tissue or organ being treated.
2) Liquid particles, on the other hand, can transport pharmaceutical agents, non-pharmaceutical agents, especially excipients, together and permit a depot effect during optimization of the effect location. However, such liquid aerosols from complex substance mixtures often have stability problems that can adversely affect the efficacy of the agent in the lungs or in the system. Solid particles are superior to liquid particles because of their high stability and can be produced with present release methods (atomization or pulverization) over a broad size range. The penetration depth of the particles into the lungs can therefore be deliberately controlled, as in liquid particles. Particles in the size range of 1.5 μm and smaller can reach almost any lung tissue, whereas large particles mostly penetrate into the areas of the air-conducting airways (bronchi).

One possible target location or effect location (target) of pharmaceutical agents or their formulations transferred by the inhalation is the lung itself, especially in the already mentioned medical indications for which administration of D2O as an aerosol is suitable.

Administration of an aerosol according to the invention preferably occurs via an inhaler, also called an atomizer. Any standard inhaler appropriate for medical aerosols is usable as an inhaler for the present invention. An inhaler can also be used to produce aerosols according to the invention. D2O solutions, D2O/H2O solutions, combinations according to the invention are fed to the inhaler in order to produce the preferably propellant-free aerosols from it according to the invention. The inhaler for this purpose sprays a defined volume of formulation using high pressures through small nozzles in order to generate an inhalable aerosol according to the invention. Inhalers that can atomize a small amount of a liquid D2O solution according to the invention in a therapeutically appropriate dose within a few seconds into a therapeutically inhalable appropriate aerosol are particularly suitable. Such inhalers are suitable in particular for propellant-free administration of the aerosols or pharmaceutical compositions according to the invention. One such atomizer is described for example in international patent applications WO 91/14468 and WO 97/12687. In such an atomizer a drug solution is converted by high pressure of up to 600 bar into a medical aerosol appropriate for application to the airways and lungs and sprayed. For atomization of such a solution a special nozzle is used, as described for example in WO 94/07607 or WO 99/16530.

Appropriate inhalers for aerosols according to the invention also include propellant-driven inhalers (or atomizers). Propellants in this case can be CFCs or HFCs. “Theory and Practice of Inhalation Therapy”, pages 31-70, Arcis Verlag (2000) is referred to in this respect, where a detailed description of usable atomizers and methods for their use is/are disclosed.

Further examples for appropriate inhalers are compressed air-driven nozzle atomizers (for example, PARI LC plus, PARI GmbH, Starnberg, Germany), Venturi nozzle atomizers, water vapor-driven nozzle atomizers or ultrasound atomizers (for example, AeronebLab, Aerogen, Inc., Stierlin Court, Canada; eFLOW, PARI GmbH, Starnberg, Germany). Inhalers with a size that can be carried along by the patient (person) are also suitable, for example, the Respimat® as described in WO 97/12687. A detailed description of appropriate inhalers can also be found in “Theory and Practice of Inhalation Therapy”, pages 31-70, Arcis Verlag (2000). All references mentioned in this description of the present invention are fully included in the present invention.

A further preferred form of administration of the present invention is administration of D2O via an endoscope (bronchoscopy). This preferred administration of D2O leads to an increase in the amount of D2O available in the alveoli. Such an increase can be necessary under the following conditions:
1) The processes of inhalation and exhalation limit the maximum amount of D2O that could be fed to the alveoli by inhalation. This can lie below the amount deemed necessary for certain therapeutic objectives,
2) By accumulation of cells as a result of fibrosis or a lung tumor, lung areas can already be largely excluded from gas exchange, in which case D2O administered as aerosol would not effectively reach the alveoli located there.

In these cases a preferred direct contact of D2O as liquid occurs according to the invention with parts of the lung surface by filling of D2O into parts of the lungs (for example, by means of an endoscope). Endoscope is understood to mean any appropriate device for administration of D2O to the lungs of a mammal.

In the context of the present invention the term endoscope is to be understood to be synonymous with the term bronchoscope. Filling of the lungs with water or with aqueous solutions and their later removal is known in the prior art and is used in certain indications mostly in the context of bronchoscopy for rinsing of the lungs (bronchoalveolar lavage, BAL). According to the present invention the D2O is filled into parts of the lungs, preferably via an endoscope, so that the alveoli in this area are largely filled with D2O. The D2O is then left in the lungs for a time appropriate for therapy, for example 2 to a maximum of 36 hours and then removed again. D2O application, if necessary, can be repeated at time intervals over several months, preferably 2 months, especially 3 months and more especially 6 months and even more especially 12 months. The advantage of this procedure referred to subsequently as D2O-BAL is the direct contacting of the alveolar surface with D2O and therefore maximum penetration of D2O.

Preferred variants of D2O-BAL according to the invention represent applications of a D2O solution according to the invention with the following additions or modifications, the effects of which are described. It is understood that this list is not definitive:
1) Mixing of H2O with a D2O solution according to the invention. The optimal amount of D2O for the therapeutic purpose can be adjusted by this,
2) Use of a D2O solution according to the invention with the highest possible D2O concentration (preferably D2O with an isotope purity greater than 95%), which causes correspondingly high enrichment of D2O in selected diseased lung areas. This procedure can be combined with adding appropriate molecules to the D2O solution that alter the osmotic conditions on the alveolar interface significantly in one direction without, however, causing osmotic shock. In this case the mixture of cellular water (from the cytoplasm of the alveolar epithelial layer) would generate heat with the alveolar D2O, which would be released directly at the inter face.
3) Addition of substances that are suitable for changing the osmotic conditions between the applied D2O solution according to the invention and the alveoli. Control of entry of D2O into the cells and emergence of cellular H2O into the alveolar D2O solution can therefore occur.

According to the invention the D2O used for D2O-BAL can preferably also be used as a combination according to the invention. Preferred additional pharmaceutical or further non-pharmaceutical agents suitable for this purpose include the agents already described above in detail. The concentrations of D2O and of the pharmaceutical or non-pharmaceutical agents pertain to the already mentioned concentration information.

A further preferred variant of the present invention concerns use of deuterium oxide, in which deuterium oxide is administered as a formulation.

A further object of the present invention is therefore a D2O-containing formulation.

Such a formulation according to the invention is preferably a ointment, a cream, a lotion, an emulsion or a gel or hydrogel.

Ointment according to the present invention is understood to mean a drug preparation to be used externally from a base of lubricating substance, like Vaseline to which the actual pharmaceutical and/or non-pharmaceutical agents are added, for example, by mixing.

Cream according to the present invention is understood to mean a ointment that can contain additional ingredients, like cosmetic agents, for example, fragrances, dyes and/or emulsifiers, for example, lecithin. A lotion can be distinguished from a cream in general, this distinction mostly being made as a function of degree of viscosity. Cream according to the invention is also understood to mean a lotion.

Emulsion according to the present invention is understood to mean a macro- or microemulsion, either on a water-in-oil or oil-in-water basis.

Gel according to the present invention is the solution of a macromolecular substance, for example, agarose, acrylic acid, alginic acid, polysiloxanes or acrylamide, whose concentration is so high that the dissolved macromolecules are combined to a sponge-like three-dimensional framework under appropriate conditions and optionally with addition of other substances (for example, salts, acids, fillers, buffers), in whose cavities a liquid is found. Gels have relatively firm consistency on this account. The viscosity lies between liquid and solid. Such a liquid is preferably pure D2O or a mixture of D2O and H2O.

A gel characterized by particularly high absorption capacity of water is referred to as hydrogel according to the invention consisting of preferably 20 to 99% according to the invention, more preferably 70 to 99% and especially 80 to 99% water without, however, exhibiting the rheological properties of a conventional liquid. In a particularly preferred variant the hydrogel is transparent and at the same time spreadable without adversely affecting its morphology and integrity by spreading of the gel.

Preparation of a formulation usable according to the invention, especially a ointment, cream, lotion, emulsion or a gel or hydrogel is described as in the examples. If such a formulation contains additional pharmaceutical and/or non-pharmaceutical agents; these are preferably added through the mixing of the formulation. However, it can occur according to any standard methods known in the prior art. Such methods are known to one skilled in the art as are the concentrations of the components or substances to be used.

The concentrations of D2O in the usable formulation according to the invention preferably lie in the following ranges:
for a cream or ointment preferably in the range from 0.1 to 95 wt %, especially from 5 to 85 wt %, also especially from 10 to 80 wt %, with particular preference from 15 to 70 wt % and even more preferably from 20 to 60 wt % and most preferably from 25 to 55 wt % and
for an emulsion preferably in the range from 1 to 98%
for a gel or hydrogel preferably 0.1 to 99.8 wt %, especially 10 to 99 wt %, also especially from 15 to 80 wt %, with particular preference from 20 to 70 wt %, more preferably from 30 to 70 wt % and most preferably from 35 to 65 wt %.

One skilled in the art will choose the appropriate concentration, depending on the present indication, the condition of the organism (patient) being treated, the severity of the disease, etc.

In a particularly preferred variant a formulation usable according to the invention also contains at least one inorganic or organic solvent. The solvent is preferably selected from the group consisting of ethanol, water and glycerol as well as their mixtures.

An organism being treated according to the present invention is an animal organism, especially a vertebrate, especially a mammal, particularly a human, horse, pig, cow, goat, sheep, cat and dog.

The present invention is further explained below by means of examples and figures, in which these do not restrict the objects of the invention.

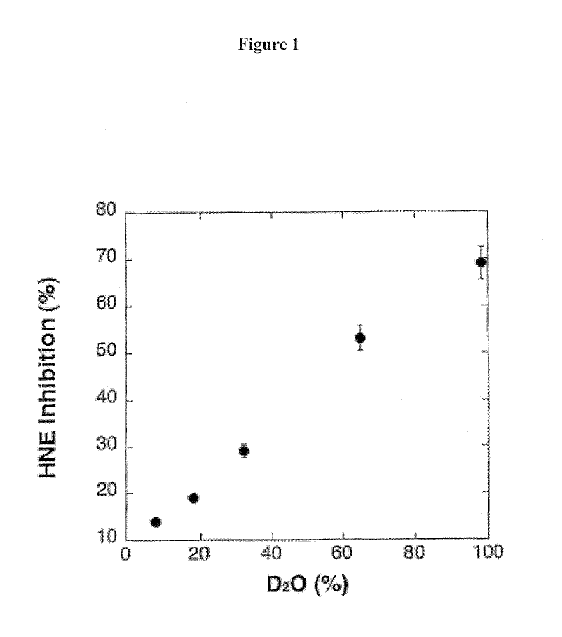

FIG. 1 shows inhibition of conversion of the synthetic substrate N-methoxysuccinyl-Ala-Ala-Pro-Val-p-nitroanilide (AAPV) by human neutrophil elastase (HNE) as a function of the volume fraction of D2O in the mixture of HNE and AAPV. Measurement of AAPV conversation occurred spectrophotometrically at a wavelength of 405 nm.

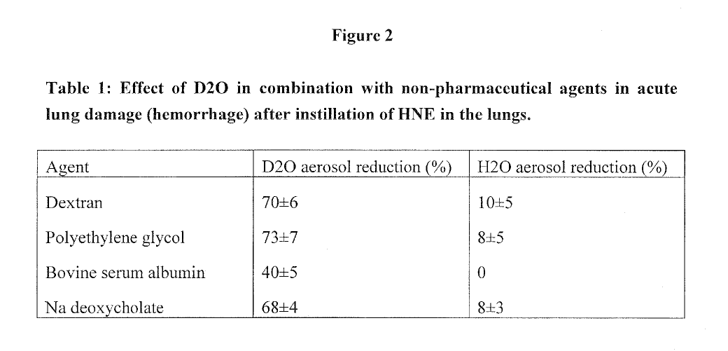

FIG. 2 shows Table 1 and the acute lung damage (hemorrhage) after instillation of serine protease elastase into hamster lung, determined by spectrophotometric determination of hemoglobin concentration in the wash solution after BAL. The percentage reduction (% reduction) of acute lung damage by D2O and H2O aerosols which were provided with different additional non-pharmaceutical agents (referring to the control group not treated with aerosol) is shown. The concentration of the non-pharmaceutical agent in the aerosol was 1 wt % in each case. The percent reduction was calculated from the reduction of hemoglobin concentration in the wash solution.

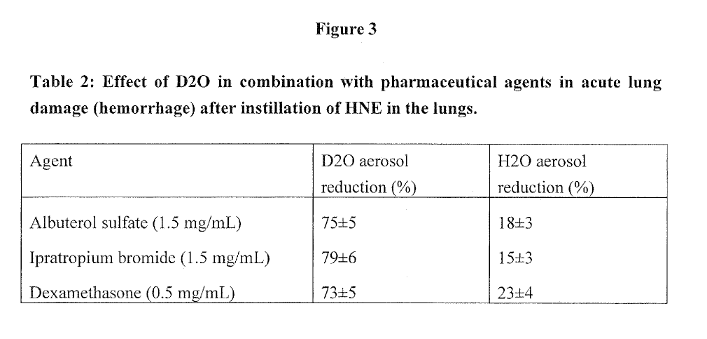

FIG. 3 shows Table 2 and the acute lung damage (hemorrhage) after instillation of serine protease elastase into hamster lung, determined by spectrophotometric determination of hemoglobin concentration in the wash solution after BAL. The percentage reduction (% reduction) of acute lung damage by D2O and H2O aerosols provided with different additional pharmaceutical agents is shown (referring to the control group not treated with aerosol). The concentration of additional pharmaceutical agent in the aerosol is shown in parentheses.

The percent reduction was calculated from the reduction of hemoglobin concentration in the wash solution.

### EXAMPLES

The D2O used for all examples (CU Chemie, Uetingen, Switzerland) had an isotope purity of 98%. The employed H2O was distilled and ion-exchanged. Both D2O and H2O were sterile.

### Example 1

### Preparation of Hydrogels Based on Acrylic Acid

2.0 wt % Carbopol 980 (manufacturer: Noveon, Inc., 9911 Brecksville Rd., Cleveland, Ohio 44141-3247, USA) was dissolved in separate charges in pure D2O, in pure H2O or in a mixture of D2O and H2O by agitation and then titrated to a pH value of 6.8 by pipetting of 10M NaOH solution. The colorless transparent and optically clear acrylic acid gels (Carbopol gels) (D2O Carbopol gel, H2O Carbopol gel, D2O/H2O Carbopol gel) that formed by NaOH addition as a result of crosslinking of the polyacrylic acid via its carboxyl groups with the alkali hydroxyl groups was then stored at room temperature until further use for at least 24 hours.

### Example 2

### Preparation of Hydrogels Based on Siloxanes (Silicone)

3.0 wt % hexamethyldisiloxane (commercial name SILMOGEN CARRIER from Dow Corning) and 1 wt % ethanol were dissolved in separate charges in pure D2O, in pure H2O or in a mixture of D2O and H2O by agitation. These solutions were then mixed immediately in a weight ratio of 1:2 (silicone solution: Carbopol gel) by vigorous agitation with the gel (Carbopol gel) prepared according to example 1 until optically transparent gels (silicone gels) (D2O-silicone gel, H2O-silicone gel, D2O/H2O-silicone gel) formed. Storage of the gels occurred at room temperature until further use for at least 24 hours.

### Example 3

### Preparation of Hydrogel Based on Alginates

4.0 wt % alginic acid sodium salt (Na alginate) (manufacturer: Röhm GmbH, Darmstadt, Germany) was dispersed in separate charges in pure D2O, in pure H2O or in a mixture of D2O and H2O and then titrated to a pH value of 7.0 by pipetting of 10M NaOH solution. The yellowish brown transparent gels (alginate gels) (D2O-alginate gel, H2O-alginate gel, D2O/H2O-alginate gel) formed were stored at room temperature until further use for at least 24 hours.

### Example 4

### Preparation of Hydrogen Based on PVA

20 wt % polyvinyl alcohol (PVA C-25, Shin-Etsu Chemical Co., Japan) was dissolved in separate charges in pure D2O, in pure H2O or in a mixture of D2O and H2O by agitation. The solutions were then subjected to five freezing-thawing cycles. The results were gels (PVA gels) (D2O-PVA gel, H2O-PVA gel, D2O/H2O-PVA gel) with rubber-like properties which were cut into 2 mm thin disks. The gels were stored at room temperature until further use for at least 24 hours.

### Example 5

### Preparation of Hydrogel Films or Plates Based on Agarose

3.0 wt % agarose in separate charges was dissolved in pure D2O, in pure H2O or in a mixture of D2O and H2O and the solutions then heated to 90° C. The employed D2O from Sigma-Aldrich (Munich, Germany) had an isotope purity of 98.5%. The hot solutions were poured into appropriate petri dishes to a height of 1.0-1.5 mm and cooled. The gels so obtained (agarose gels) (D2O-agarose gels, H2O-agarose gels, D2O/H2O-agarose gels) were stored under sterile conditions at 4° C.

### Example 6

### Preparation of Hydrogel Films or Plates Based on Acrylamide

Acrylamide gels (5% acrylamide) were prepared in which pure D2O, pure H2O or a mixture of D2O and H2O in separate charges were degassed before addition of acrylamide (with 2.4% bis-acrylamide) and heated to 40° C. After addition of acrylamide and bis-acrylamide the solutions were mixed (Vortex mixer, 1 minute at 200 rpm) and the catalysts tetramethylethylenediamine (TEMES; 1.0%) and ammonium persulfate (AP; 0.1%) were added followed by 10 seconds of mixing. The gels were then poured into petri dishes (height of the gel 1.0-1.5 mm) and stored for 2 hours at 40° C. The gels (D2O-acrylamide gel, H2O-acrylamide gel, D2O/H2O-acrylamide gel) were then washed, in which the similar water mixture as for the hydration of the gel (pure D2O, pure H2O or a mixture of D2O and H2O) was used for washing. The gels were stored at room temperature until further use for at least 24 hours.

### Example 7

### Preparation of a D2O-Containing Cream

D2O was slowly added to 50 grams of Asche basic cream (manufacturer: Asche Chiesi GmbH, Hamburg, Germany) at 40° C. during continuous agitation until a weight fraction of 38% D2O (referring to the initial weight of the cream) was reached in the homogeneous mixture. The cream was then cooled to room temperature and stored closed airtight.

### Example 8

### Generation of D2O Aerosols

The D2O used for all examples (CU Chemie, Uetingen, Switzerland) had an isotope purity of 98%. The employed H2O was distilled and ion-exchanged. Both D2O and H2O were sterile. A Pari LC Plus universal atomizer (PARI GmbH, Starnberg, Germany) was used for aerosolization combined with a Pari universal compressor, which generated 200 mg/min polydispersed aerosol with an average particle size (median diameter) of 2.5 μm for pure H2O and pure D2O and 2.5-4.5 μm for H2O and D2O with additional non-pharmaceutical and/or pharmaceutical agents (operating pressure 2.0 bar, flow rate of the compressor air was 6.0 L/min). The particle size measurement occurred with dynamic light scattering in a flow cell. Aerosol generation occurred at a temperature of 37° C. by corresponding thermostating of the atomizer in a water bath thermostat.

### Example 9

### Inhibition of the Enzyme Human Neutrophil Elastase (HNE)

HNE was incubated together with the synthetic substrate N-methoxysuccinyl-Ala-Ala-Pro-Val-p-nitroanilide (AAPV). Conversion of AAPV was then determined photometrically at 405 nm. The following incubation scheme was used:
Sample 1) HNE IN D2O with AAPV in D2O;
Sample 2) HNE in D2O with AAPV in H2O;
Sample 3) HNE in H2O with AAPV in D2O;
Control: HNE in H2O with AAPV in H2O.

The enzyme and substrate were each incubated for 60 minutes with H2O or D2O before HNE and AAPV were added together. The amount of D2O in the experiments was varied over a range from 10 to 100% in order to detect the dependence of inhibition of AAPV conversion by HNE.

FIG. 1 shows inhibition of AAPV conversion as a function of D2O amount (vol %) in the mixture. A half-maximum effective dose was determined at a D2O content of about 40 vol %. At 100% D2O in the mixture an inhibition of AAPV conversion of about 70% was achieved.

### Example 10

### Effectiveness of D2O Instillation in an Animal Model of COPD

To investigate the effect of D2O in a model for chronic obstructive pulmonary disease (COPD) male Syrian Gold hamsters (average weight 118 g±4 g) were divided into two groups (experimental group and control group) of 10 hamsters each. The animals of both groups were initially anesthetized intraperitoneally (ketamine 95 mg/kg and xylazine 17 mg/kg) and then intubated endotracheally with direct laryngoscopy. The experimental group received 250 μL of 0.9% NaCl solution based on D2O (98.5% deuterium enrichment) instilled, whereas 250 μL of a corresponding isotonic NaCl solution based on H2O was used in the control group. Each animal received after the end of this instillation a second instillation at a time interval of 30 minutes, which was identical for both groups: 100 μL of human neutrophil elastase (HNE, 500 μg/mL in sterile 0.9% NaCl solution). The animals were killed 4 hours (six animals per group) or 24 hours (four animals per group) after the last instillation (phenobarbital 70 mg/kg intraperitoneally).

For the animals killed 4 hours after instillation a BAL (bronchoalveolar lavage) was performed. 2.5 mL 0.9% NaCl solution was repeatedly instilled (total 3 times) for BAL and the hemoglobin concentration in the instillate then determined spectrophotometrically. The obtained value was used as a gauge for lung damage (hemorrhage) by human neutrophil elastase (HNE). For the control group an average hemoglobin value of 320±50 mOD (milli-optical density) was obtained. For the experimental group an average hemoglobin value of 170±20 mOD was measured. This reduction in hemoglobin concentration in BAL corresponds to inhibition of the acute lung damage caused by elastase (hemorrhage) of 47%.

For the animals killed 24 hours after instillation the lungs were removed, fixed in formalin and embedded in paraffin. Histologic longitudinal sections of the lungs in the direction of the lung hilus were prepared, stained and made visible with iron by means of an histochemical reaction (Berlin blue reaction), which serves as a gauge for the intensity of HNE-induced hemorrhage. The microscopic investigation of the sections gave the following result:

Control group: high number of blue-stained cells (macrophages, which had internalized blood residues) as an expression of pronounced hemorrhage.

Experimental group: significantly smaller number of blue-stained cells as an expression of more limited hemorrhage.

### Example 11

### Efficacy of D2O Aerosols in an Animal Model of COPD

Male Syrian Gold hamsters (average weight 105 g±8 g) were divided into two groups (experimental group and control group) of six hamsters each. The animals of both groups were anesthetized intraperitoneally (ketamine 95 mg/kg and xylazine 17 mg/kg) and then intubated endotracheally by direct laryngoscopy. Both groups received 100 μL of human neutrophil elastase (500 μg/mL in sterile 0.9% NaCl solution) instilled. The animals were then brought to separate cages and subjected to continuous treatment with D2O aerosol (experimental group) or H2O aerosol (control group) (prepared according to example 8) in the respiration air (humidity 60%) for 24 hours. After this treatment the animals were killed (phenobarbital 70 mg/kg intraperitoneally) and a BAL (bronchoalveolar lavage) carried out. For BAL 2.5 mL 0.9% NaCl solution was repeatedly instilled (total of three times) and the hemoglobin concentration then determined in the instillate speetrophotometrically. The value so obtained was used as gauge for lung damage (hemorrhage) by human neutrophil elastase (HNE). For the control group an average hemoglobin value of 300±50 mOD (milli-optical density) was obtained. For the experimental group an average hemoglobin value of 120±20 mOD was measured. The reduction in hemoglobin concentration in the experimental group corresponds to an inhibition of acute lung damage caused by elastase (hemorrhage) of 60%.

### Example 12

### Demonstration of Efficacy of D2O Aerosols Mixed with Non-Pharmaceutical Agents on an Animal Model of COPD

Male Syrian Gold hamsters (average weight 105 g±8 g) were divided into two groups (D2O and H2O group) of eight hamsters each. The two groups were then divided into subgroups of two hamsters each, i.e., four D2O groups and four H2O groups. A third group (control group) of two hamsters was additionally created. The animals of all three groups were first anesthetized intraperitoneally (ketamine 95 mg/kg and xylazine 17 mg/kg) and then intubated endotracheally by direct laryngoscopy. Both groups received 100 μL of human neutrophil elastase (500 μg/mL in sterile 0.9% NaCl solution with H2O) instilled. The animals of the D2O and H2O groups were then introduced to separate cages (two hamsters per cage each) and subjected to continuous treatment with D2O aerosol (D2O group) or H2O aerosol (H2O group) (prepared according to example 8) in the respiration air (humidity 60%) for 4 hours. The D2O and H2O aerosols contained one of the following substances with the concentrations given in parentheses in the initial solution used for its generation according to example 8 (i.e., the solution before aerosolization): dextran 4000 (1.0 wt %), polyethylene glycol 4000 (1.0 wt %), bovine serum albumin (1.0 wt %), sodium deoxycholate (1.0 wt %). The animals were treated with the corresponding aerosols (D2O or H2O) for 4 hours, the relative humidity in the cage was then 60%. After this treatment all animals (including the two animals of the control group not treated with aerosol) were killed (phenobarbital 70 mg/kg intraperitoneally) and a BAL (bronchoalveolar lavage) carried out. For BAL 2.5 mL 0.9% NaCl solution was repeatedly instilled (total of three times) and the hemoglobin concentration then determined in the instillate spectrophotometrically. The value so obtained was used as gauge for lung damage (hemorrhage) by human neutrophil elastase (HNE). Table 1 gives the percent reduction of acute lung damage caused by HNE calculated from the corresponding hemoglobin concentration.

### Example 13

### Demonstration of Efficacy of D2O Aerosols Mixed with Additional Pharmaceutical Agents on the Animal Model of COPD

As additional pharmaceutical agents the beta-agonist albuterol (albuterol sulfate) and the anticholinergic ipratropium (ipratropium bromide) from the group of bronchodilators and the glucocorticoid dexamethasone selected from the group of steroids were tested in combination with D2O aerosols relative to the efficacy for treatment of COPD. Male Syrian Gold hamsters (average weight 105 g±8 g) were divided into two groups (D2O and H2O group) of six hamsters each. The two groups were then divided into three subgroups of two hamsters each, i.e., three D2O groups and three H2O groups. A third group (control group) of two hamsters was additionally created. The animals of all three groups were initially anesthetized intraperitoneally (ketamine 95 mg/kg and xylazine 17 mg/kg) and then intubated endotracheally by direct laryngoscopy. Both groups received 100 μL of human neutrophil elastase (500 μg/mL in sterile 0.9% NaCl solution) instilled. The animals of the D2O and H2O groups were then introduced to separate cages (two hamsters each per cage) and subjected to continuous treatment with D2O aerosol (D2O group) and H2O aerosol (H2O group) (prepared according to example 8) in the respiration air (humidity 60%) for 4 hours. The D2O and H2O aerosols contained one of the following substances with the concentrations given in parentheses in the initial solution used for its generation according to example 8 (i.e., the solution before aerosolization): albuterol sulfate (1.5 mg/mL), ipratropium bromide (1.5 mg/mL), dexamethasone (0.5 mg/mL). The animals were treated with the corresponding aerosols (D2O or H2O) in the respiration air for 4 hours, the relative humidity in the cage was 60%. After this treatment all animals (including the two animals of the control group not treated with aerosol) were killed (phenobarbital 70 mg/kg intraperitoneally) and a BAL (bronchoalveolar lavage) carried out. For BAL 2.5 mL 0.9% NaCl solution was repeatedly instilled (total of three times) and the hemoglobin concentration in the instillate then determined spectrophotometrically. The value so obtained was used as gauge for lung damage (hemorrhage) from serine protease (elastase). Table 2 shows the percent reduction of acute lung damage caused by HNE calculated from the corresponding hemoglobin concentration.

### Example 14

### Determination of Efficacy of D2O for Inhibition of Inflammation in Myocardial Tissue

In an animal model for reperfusion damage a reduced blood supply to the heart muscle tissue was achieved by ligature of a coronary artery in male white New Zealand rabbits, which led to damage (Bidouard, J. P. et al.; SSR69071, an elastase inhibitor, reduces myocardial infarct size following ischemia reperfusion injury, Eur J Pharmacol. 2003; 461:49-52). By thoracotomy a side branch of the left coronary artery was closed by ligature for 30 minutes and then opened again in order to achieve reperfusion over 120 minutes. Before removal of the heart an ink bolus (pelican ink) was introduced to the ascending aorta. To investigate the effect of D2O on reperfusion damage the animals were divided into three groups of two animals each. Two groups received an intravenous treatment with D2O in the form of isotonic saline either 15 minutes before (experimental group 1) or 25 minutes after (experimental group 2) reopening of the coronary artery, the other group (control group) was treated with H2O as isotonic saline. After removal of the heart, unclamping of the aorta, formalin fixation and paraffin embedding the heart was investigated for homogeneity of ink distribution in the capillary system through three cross sections and by immunohistochemistry. The immunohistochemical detection of granulocytes occurred by a-naphthol chloroacetate esterase staining. In the animals of the control group a significantly higher content of granulocytes and much lower capillary marking by pelican ink was found. Histologic differences between experimental groups 1 and 2 were not significant. It can therefore be demonstrated that significantly lower heart muscle damage occurred in the D2O-treated groups than in the H2O-treated animals.

### Example 15

### Demonstration of Efficacy of D2O Inhibition of Inflammation in the Brain After Cerebral Ischemia

The effect of D2O on cerebral ischemia was investigated in an animal model. For this purpose cerebral ischemia was produced for 1 hour in adult Long-Evans rats by closure of both carotid arteries, which followed reperfusion over 24 hours. Final observation with evaluation of neurovegetative symptoms occurred (spontaneous activity, coordinated walking, stretching of the front paw and climbing). Damage to the brain tissue was then investigated histologically. In two experimental groups of three rats each, either D2O (experimental group) or H2O (control group) in isotonic saline was infused in the animals 15 minutes before initiation of ischemia and right before subsequent reperfusion.

A significant effect on the reduction in reperfusion damage (histological demonstration similar to example 10) was only found in the groups treated with D2O both in terms of the presence of granulocytes in histological sections and also neurological behavior of the animals after the operation.

### Example 16

### Efficacy of D2O in Bullous Pemphigoid

The blister fluid in patients with bullous pemphigoid was sampled in an ex vivo model (Verraes et al., 2001). Recombinant radioactively-labeled BP180 antigen, the target antigen in this disease, was then added to the blister fluid and decay determined autoradiographically after gel electrophoresis. For inhibition of elastase activity in the blister fluid either D2O or the elastase inhibitor chloromethyl ketone was added to it. An untreated control served as comparison. Both with chloromethyl ketone and with D2O the degradation of the recombinant BP180 antigen could be inhibited, whereas the protein was almost completely degraded by the untreated blister fluid.

### Example 17

### Efficacy of D2O in Psoriasis Vulgaris

A half-side experiment was conducted by means of hydrogel to determine the effect of D2O in psoriasis vulgaris. The gel was a 2 wt % Carbopol 980 with 1 wt % urea. The pH value of the gel was set at 6.5 with NaOH solution. Patients having comparable lesions on both elbows were treated. The local psoriasis severity index (LPSI) was used for evaluation (Henneicke-von Zepelin, H. H., Mrowietz, U., Färber, L., Bruck-Borchers, K., Schober, C., Huber, J., Lutz, G., Kohnen, R., Christophers, E., Welzel, D., Highly purified omega-3-polyunsaturated fatty acids for topical treatment of psoriasis. Results of a double-blind, placebo-controlled multicenter study. Br J Dermatol. 1993; 129:713-7). The patients were relied on to apply the content of tubes marked “right” and “left” twice a day on the corresponding lesions on the right or left elbow. A gel produced with D2O was found in one tube, the gel in the other tube was prepared with H2O. Division occurred randomly. LPSI was determined once a week. After 4 weeks of treatment and allocation of the applications to corresponding sides, a significant reduction of LPSI was found in the D2O-treated lesions in comparison with those to which H2O gel was applied.

### Example 18

### Efficacy of D2O in Peritonitis and Septic Shock

The effect of D2O for application in peritoneal dialysis was investigated in an animal model of infectious peritonitis (Welten, A. G., Zareie, M., van den Born, J., ter Wee, P. M., Schalkwijk, C. G., Driesprong, B. A., Mul, F. P., Hordijk, P. L., Beelen, R. H., Hekking, L. H., In vitro and in vivo models for peritonitis demonstrate unchanged neutrophil migration after exposure to dialysis fluids. Nephrol Dial Transplant. 2004; 19:831-9). Male Wistar rats received a catheter for peritoneal dialysis and for induction of peritonitis and for further peritoneal dialysis. A purulent peritonitis was produced by introduction of a suspension of S. aureus ATCC 25923 (1×10⁹ c.f.u. in 0.5 mL). After 4 hours peritoneal lavage was carried out either with isotonic saline or with isotonic saline 30%/H2O 70%. After the death of the animals, but at the latest after 24 hours, bacterial infestation and the inflammatory reaction in the peritoneum were investigated histologically. Both a reduced number of bacteria in the quantitative culture and lower inflammatory activity in the animals treated with D2O were found.

### Example 19

### Efficacy of D2O in Allergic Diseases

In one experiment an extract of the house dust mite Dermatophagoides pteronyssinus (D. pter) was lyophilized and taken up in D2O. The D. pter/D2O extract was then investigated in a Prick test in patients who had reacted positively to the conventional test liquid with D. pter. The untreated test solution was used again for control, histamine solution 0.1% which was prepared with H2O, histamine solution 0.1% which was prepared with D2O and isotonic saline. It was found that during use of D2O/D. pter test solution only a very limited wheal reaction could be triggered, whereas the wheal reaction with the untreated D. pter solution corresponded roughly to the histamine control. No differences were found between the D2O and H2O-histamine prick tests.

A solution with D2O was prepared accordingly for application as nasal spray. Five patients each with demonstrated type 1 sensitization and allergic rhinitis were treated either with the D2O-containing nasal spray (isotonic saline using 70% D2O) or with isotonic saline with H2O. The number of sneezing attacks was determined and the intensity of runny nose and nasal itching measured by visual analog scales as parameters. For this purpose a patient diary was used. After an experiment time of 14 days and use of corresponding sprays 6 times a day a significant reduction of the recorded parameters was found in the group treated with D2O in comparison with the H2O group.

### Example 20

### Efficacy of D2O for Decongestion of Nasal Mucosa

Physiological saline was prepared from D2O (experiment group) and H2O (control group) and filled into a pump spray container as is ordinarily used to release nasal spray. Subjects with colds accompanied by severe nasal obstruction were divided into two groups of eight subjects each. Air flow through the nose was determined by rhinomanometry (initial value) in each subject right before treatment (after thorough removal of secretions). The subjects of the experimental group then each received one pump spray of D2O saline in each nostril, while the subjects of the control group received H2O saline instead in the same amount. Thirty and 60 minutes after application air flow was determined in each subject by rhinomanometry and the percent change of air flow relative to the initial value was calculated. From these values an average was formed for the verum and control groups (average percentage change in air flow <L_{f}%>). A positive value of <L_{f}%> indicates an increase, a negative value a reduction in air flow. For the experimental group after 30 minutes a value of <L_{f}%>=20±5% and after 60 minutes <L_{f}%>=90±10% was obtained. The control group gave <L_{f}%>=10±5% after 30 minutes and <L_{f}%>=5±5% after 60 minutes. A significant increase in air flow and therefore a related decongestion of the nasal mucosa could be demonstrated for the experimental group (D2O) compared to the control group (H2O).

### Example 21

### Efficacy of D2O for Treatment of Acute Inflammation of the Nasal Sinuses (Sinusitis)

Aerosols from D2O and H2O each mixed with 150 mM NaCl were prepared according to example 2. Subjects with acute sinusitis in the maxillary sinus area were divided into two groups. The experimental group inhaled the D2O aerosol for 15 minutes, the control group the H2O aerosol. During inhalation the subjects were relied on to inhale through the nose as much as possible. Every 3 hours after inhalation a rhinoendoscopic examination of the sinuses was conducted. A significant reduction of mucosal swelling was found in the experimental group compared to the control group. This led to improved outflow of secretion in the experimental group.

### Example 22

### Efficacy of D2O in Allergic Rhinitis

A solution with D2O was prepared according to example 21 for use as nasal spray. Five patients each with demonstrated type 1 sensitization and allergic rhinitis were treated either with the D2O-containing nasal spray (isotonic saline using D2O 70%) or with isotonic saline with H2O. The number of sneezing attacks was determined as parameter and the intensity of runny nose and nasal itching were measured by visual analog scales. For this purpose a patient diary was used. After an experiment time of 14 days and use of corresponding spray 6 times a day a significant reduction of the recorded parameters was found in the D2O-treated group in comparison with the H2O group.

### Example 23

### Efficacy of D2O in Allergic Conjunctivitis

A corresponding animal model was used to test the efficacy of eye drops prepared with D2O (Minami et al., Biol. Pharm. Bull 28:473-476, 2005). In 6-week-old Wistar rats an allergy to chicken protein was produced by injection of chicken protein together with alum and 10¹⁰ B. pertussis into the soles of the feet and then a booster with chicken protein in the back area. Before the experiment the animals were kept in a climatized cage and after 10 minutes of adaptation 5 μL chicken protein solution was trickled into each eye. The animals were then transferred to an observation cage and the scratching movements in the direction of the eyes counted over a period of 20 minutes. Hyperemia and conjunctival edema were then determined.

To investigate an effect of D2O on the symptoms caused by chicken protein either H2O- or D2O-based isotonic saline was trickled together with the chicken protein in a double-blind design. After 20 minutes a significant reduction in the number of scratching movements in the eye direction was found in the D2O-treated group in comparison with the H2O-treated animals. Hyperemia and edema were also reduced in the D2O group in comparison with the H2O group. It could be concluded from these experiments that the D2O-containing eye drops are capable of significantly reducing the symptoms of allergic conjunctivitis.

### Example 24

### Efficacy of D2O in Allergic Bronchial Asthma

The effect of D2O in allergic asthma was presented in an animal model of 10-week-old brown Norwegian rats (Roumestan et al., Resp Res 8:35-46, 2007). Sensitization of the animals occurred by repeated intraperitoneal injection of ovalbumin. The animals were then introduced to a climatized cage and constantly treated with an aerosol of isotonic H2O or D2O solution for 6 days. Asthma triggering by an ovalbumin-containing aerosol then occurred. The change in airway resistance was then determined as parameter by barometric plethysmography on the living animals after 24 hours. A significant intensification of airway resistance was found in the H2O-treated group. In the animals treated with D2O aerosol only a slight increase in airway resistance occurred. These data suggest the conclusion that D2O can find application in the treatment of allergic bronchial asthma.

### Example 25

### Efficacy of D2O-Containing Gel After Topical Administration on Progress of Leukocytoclastic Skin Vasculitis

Leukocytoclastic skin vasculitis is characterized by recurring behavior. A special feature of this dermatosis is destruction of cutaneous capillaries by an inflammatory process in which neutrophil granulocytes participate almost exclusively. To check the effect of D2O-containing gel on the extent of cutaneous lesions patients with leukocytoclastic skin vasculitis were treated in a half-side experiment. Either an H2O- or D2O-containing gel were applied in randomized fashion on the right or left lower leg three times a day. After a treatment time of 4 days the treatments was compared by means of a clinical score that evaluates the number and expression of vasculitis lesions. A distinct reduction of the clinical score was then found on the lower legs that had been treated with D2O gel.

### Example 26

### Efficacy of D2O Infusions in Systemic Vasculites on the Example of Wegener's Granulomatosis

Activation of neutrophil granulocytes is assigned importance in the pathogenesis of Wegener granulomatosis. The disease activity can be determined by determining proteinase 3-antineutrophil cytoplasmic antibodies (PR3-ANCA).

In patients with Wegener's disease in an open study the effect of additional infusion of 500 mL of an isotonic saline solution prepared with 70% D2O was investigated once a day over a week in addition to individual systemic therapy in five patients. Determination of PR3-ANCA in the serum before and after 1 week after the last D2O infusion served as control parameter. Five patients in whom individual therapy was used exclusively without additional infusions served as comparison. It was then found in the patients who additionally received D2O infusions for individual therapy that there was a stronger reduction of PR3-ANCA values in comparison with conventionally treated patients. This permits the conclusion that systemic use of D2O for adjunctive therapy of systemic vasculites is suitable.

### Example 27

### Efficacy of D2O for Treatment of Recurring Benign Aphthosis (RBA)

Eight volunteer subjects with the minor form of RBA (diameter of round to oval erosion is 3-5 mm) at locations of the oral mucosa readily accessible to optical inspection (and especially cheek mucosa and mucosa in the lower jaw area) were randomized into two groups of four subjects each (experimental group and control group) and the size (diameter) of the aphthae (initial value) was determined precisely at 0.5 mm optically. For size determination only the gray-covered eroded surface was considered and the red edge area was not considered. The first aphthosis symptoms of the subjects in both groups were at least 2 days old and at most 4 days old. The experimental group received a D2O gel prepared according to example 1 three times a day applied with an appropriate applicator to the erosion (aphtha). It was ensured that the applied gel covered the entire surface of the aphtha plus a 2-3 mm wide edge and was allowed to act at least for 5 minutes. The control group was treated similarly with an identical dose and application of an H2O prepared according to example 1. Three days after the first gel application (i.e., after a total of 9 applications per subject) redetermination of the diameter of the aphthae was carried out in both groups. For the experimental group an average reduction in diameter relative to the initial value of 30±10% can be detected and in the control group an average increase in aphthae diameter by 15±10% was observed.

1-16. (canceled)

17. A method of inhibiting elastase wherein said method comprises contacting deuterium oxide with the elastase.

18. The method, according to claim 17, wherein the elastase is human neutrophil elastase (HNE).

19. The method, according to claim 17, used for prevention and/or treatment of an elastase-associated disease.

20. The method, according to claim 19, wherein the disease is selected from the group consisting of inflammatory diseases, pulmonary diseases, heart and cardiovascular diseases, and allergic diseases.

21. The method, according to claim 20, wherein the inflammatory disease is inflammation of the skin, nasal mucosa, or oral mucosa; aphthous diseases of the oral mucosa, conjunctiva, or nasal sinuses; allergic coryza; asthma; cutaneous vascularitis; pulmonary vascularitis; peritonitis or septic shock.

22. The method, according to claim 21, wherein the inflammatory disease is selected from the group consisting of neutrophil dermatoses, palmoplantar pustulosis, subcorneal pustulosis (Sneddon-Wilkinson's disease), autoimmune bullous dermatosis and pemphigoid.

23. The method, according to claim 22, wherein the pemphigoid is selected from the group consisting of bullous pemphigoid, pemphigoid vulgaris, pemphigoid vegetans and pemphigoid foliaceus.

24. The method, according to claim 20, in which the pulmonary disease is selected from the group consisting of chronic obstructive pulmonary disease (COPD), cystic fibrosis, chronic bronchitis, pulmonary fibrosis, acute respiratory tract syndrome, pulmonary emphysema and hemorrhage.

25. The method, according to claim 20, in which the heart disease and/or cardiovascular disease is selected from the group consisting of myocardial infarction, cerebral ischemia, heart failure and acute coronary syndrome.

26. The method, according to claim 20, in which the allergic disease is selected from the group consisting of house dust allergy, mite allergy, plant pollen allergy and allergic asthma.

27. The method, according to claim 17, in which the activity of human neutrophil elastase is inhibited.

28. The method, according to claim 17, in which deuterium oxide is used in combination with at least one other pharmaceutically active ingredient and/or at least one other nonpharmaceutically active ingredient.

29. The method, according to claim 28, in which the at least one other pharmaceutically active ingredient is selected from the group consisting of sulfonamides, antibiotics, corticoids, alkyl fluorophosphates, chloromethyl ketones, sulfonyl fluorides, trifluoromethyl ketones, boric acid esters, aldehydes, short-chain peptides, cytostatics, chemotherapeutics, and synthetic and plant active ingredients with an inflammation-inhibiting effect.

30. The method, according to claim 28, in which the at least one other nonpharmaceutically active ingredient is chosen from the group consisting of pharmaceutically-compatible inorganic or organic acids or bases, polymers, copolymers, block copolymers, simple sugars, multiple sugars, ionic and nonionic surfactants or lipids, pharmacologically acceptable salts, flavorings, vitamins, tocopherols, provitamins, antioxidants, stabilizers and preservatives, as well as their mixtures.

31. The method, according to claim 17, in which the deuterium oxide is administered topically, transdermally, nasally, rectally, systemically, parenterally, via perfusion, via an endoscope or as an aerosol or dry powder formulation.

32. The method, according to claim 17, in which the deuterium oxide is administered as a formulation.

33. The method, according to claim 32, in which the formulation is a salve, a cream, a lotion, an emulsion, a gel or a hydrogel.
